(19) United States
(12) Patent Application Publication
(10) Pub. No.: US 2022/0096341 A1
(43) Pub. Date: Mar. 31, 2022

(54) Array Of Anticavity Oral Care Compositions

(71) Applicant: The Procter & Gamble Company, Cincinnati, OH (US)

(72) Inventors: Arif Ali Baig, Mason, OH (US); Tammy K. Baker, Cincinnati, OH (US); Aaron Reed Biesbrock, Maineville, OH (US); Samuel James St. John, Cincinnati, OH (US)

(21) Appl. No.: 17/485,550

(22) Filed: Sep. 27, 2021

Related U.S. Application Data
(60) Provisional application No. 63/084,016, filed on Sep. 28, 2020.

(51) Int. Cl. A61K 8/21; A61K 8/9789; A61K 33/06; A61K 33/30; A61Q 11/00

(52) U.S. Cl. CPC A61K 8/21; A61K 8/9789; A61K 33/06; A61K 2800/92; A61Q 11/00; A61K 2800/28; A61K 33/30

(57) ABSTRACT

Arrays of anticavity oral care compositions with at least one anticavity oral care composition without fluoride. Arrays of anticavity oral care compositions with a first anticavity oral care composition comprising a first subtherapeutic anticaries drug and a second subtherapeutic drug where the first anticavity oral care composition provides a therapeutic anticaries benefit and is free of fluoride.

### FIELD OF THE INVENTION

The present invention is directed to arrays of anticavity oral care compositions with varying amounts of fluoride. The present invention is also directed to arrays of anticavity oral care compositions with a first oral care composition comprising a first subtherapeutic anticaries drug and a second subtherapeutic anticaries drug and a second oral care composition comprising a subtherapeutic anticaries drug comprising fluoride.

### BACKGROUND OF THE INVENTION

Oral care compositions, such as toothpaste and/or dentifrice compositions, can be applied to the oral cavity to clean and/or maintain the aesthetics and/or health of the teeth, gums, and/or tongue. Additionally, many oral care compositions are used to deliver active ingredients directly to oral care surfaces. For example, toothpaste compositions can have a fluoride ion source, such as sodium fluoride, sodium monofluorophosphate, and/or stannous fluoride, as an anticaries drug. While the effectiveness and safety of fluoride as an anticaries drug is well established, many consumers desire other options for anticavity protection.

Unfortunately, current fluoride-free oral care compositions do not provide enough, or any, protection from protection from caries caused due to the acids produced by bacteria found on the surfaces of teeth. As such, there is a need for oral care compositions that are both fluoride-free and provide an anticavity benefit.

### SUMMARY OF THE INVENTION

Disclosed herein is an array of anticavity oral care compositions, the array comprising: (a) a first anticavity oral care composition comprising: (i) a first subtherapeutic anticaries agent; (ii) a second subtherapeutic anticaries agent; and (b) a second anticavity oral care composition comprising a therapeutic anticaries agent.

Also disclosed herein is an array of anticavity oral care compositions, the array comprising: (a) a first anticavity oral care composition comprising: (i) a first therapeutic anticaries agent comprising fluoride; and (ii) a first subtherapeutic anticaries agent, wherein the first oral care composition has a therapeutic anticavity benefit greater than the first therapeutic anticaries agent; and (b) a second oral care composition comprising a second therapeutic anticaries agent, wherein the therapeutic anticavity benefit of the first oral care composition is greater than the therapeutic anticavity benefit of the second oral care composition.

Also disclosed herein is an array of anticavity toothpaste compositions, the array comprising: (a) a first anticavity toothpaste composition comprising: (i) hops; (ii) tin; and (ii) abrasive, wherein the first oral care composition is free of fluoride; and (b) a second anticavity toothpaste composition comprising: (i) fluoride; and (ii) abrasive.

Also disclosed herein is an array of anticavity toothpaste compositions, the array comprising: (a) a first anticavity toothpaste composition comprising: (i) hops; (ii) fluoride; and (ii) abrasive, wherein the first oral care composition is free of fluoride; and (b) a second anticavity toothpaste composition comprising: (i) fluoride; and (ii) abrasive.

### DETAILED DESCRIPTION OF THE INVENTION

The present invention is directed to arrays of anticavity oral care compositions with varying amounts of fluoride. While the effectiveness and safety of fluoride as an anticaries drug is well established, many consumers desire other options for anticavity protection. However, the only current active agent that can provide anticavity activity is fluoride in a therapeutic dose. Thus, a consumer has to choose between an anticavity oral care composition comprising fluoride in a therapeutic dose and other oral care compositions which do not provide a benefit that is great enough to meet the threshold to make a regulated drug claim (i.e. an “anticavity” or “anticaries” benefit).

Thus, the present invention meets this need by providing an array of anticavity oral care compositions with a varying amount of fluoride. The varying amount of fluoride can be as little as no fluoride to as much as a therapeutic amount of fluoride. Importantly, each oral care composition within the array has at least a therapeutic anticavity benefit, which will give the consumer the ability to choose their amount of fluoride without sacrificing anticavity benefit.

### Definitions

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2nd Ed (1997), can be applied, as long as that definition does not conflict with any other disclosure or definition applied herein, or render indefinite or non-enabled any claim to which that definition is applied.

The term “oral care composition”, as used herein, includes a product, which in the ordinary course of usage, is not intentionally swallowed for purposes of systemic administration of particular therapeutic agents, but is rather retained in the oral cavity for a time sufficient to contact dental surfaces or oral tissues. Examples of oral care compositions include dentifrice, toothpaste, tooth gel, subgingival gel, mouth rinse, mousse, foam, mouth spray, lozenge, chewable tablet, chewing gum, tooth whitening strips, floss and floss coatings, breath freshening dissolvable strips, or denture care or adhesive product. The oral care composition may also be incorporated onto strips or films for direct application or attachment to oral surfaces.

“Active and other ingredients” useful herein may be categorized or described herein by their cosmetic and/or therapeutic benefit or their postulated mode of action or function. However, it is to be understood that the active and other ingredients useful herein can, in some instances, provide more than one cosmetic and/or therapeutic benefit or function or operate via more than one mode of action. Therefore, classifications herein are made for the sake of convenience and are not intended to limit an ingredient to the particularly stated function(s) or activities listed.

The term “orally acceptable carrier” comprises one or more compatible solid or liquid excipients or diluents which are suitable for topical oral administration. By “compatible,” as used herein, is meant that the components of the composition are capable of being commingled without interaction in a manner which would substantially reduce the composition's stability and/or efficacy.

The term “substantially free” as used herein refers to the presence of no more than 0.05%, preferably no more than 0.01%, and more preferably no more than 0.001%, of an indicated material in a composition, by total weight of such composition.

The term “essentially free” as used herein means that the indicated material is not deliberately added to the composition, or preferably not present at analytically detectable levels. It is meant to include compositions whereby the indicated material is present only as an impurity of one of the other materials deliberately added.

While compositions and methods are described herein in terms of “comprising” various components or steps, the compositions and methods can also “consist essentially of” or “consist of” the various components or steps, unless stated otherwise.

As used herein, the word “or” when used as a connector of two or more elements is meant to include the elements individually and in combination; for example, X or Y, means X or Y or both.

As used herein, the articles “a” and “an” are understood to mean one or more of the material that is claimed or described, for example, “an oral care composition” or “a bleaching agent.”

All measurements referred to herein are made at about 23° C. (i.e. room temperature) unless otherwise specified.

Generally, groups of elements are indicated using the numbering scheme indicated in the version of the periodic table of elements published in Chemical and Engineering News, 63(5), 27, 1985. In some instances, a group of elements can be indicated using a common name assigned to the group; for example, alkali metals for Group 1 elements, alkaline earth metals for Group 2 elements, and so forth.

Several types of ranges are disclosed in the present invention. When a range of any type is disclosed or claimed, the intent is to disclose or claim individually each possible number that such a range could reasonably encompass, including end points of the range as well as any sub-ranges and combinations of sub-ranges encompassed therein.

The term “about” means that amounts, sizes, formulations, parameters, and other quantities and characteristics are not and need not be exact, but can be approximate and/or larger or smaller, as desired, reflecting tolerances, conversion factors, rounding off, measurement errors, and the like, and other factors known to those of skill in the art. In general, an amount, size, formulation, parameter or other quantity or characteristic is “about” or “approximate” whether or not expressly stated to be such. The term “about” also encompasses amounts that differ due to different equilibrium conditions for a composition resulting from a particular initial mixture. Whether or not modified by the term “about,” the claims include equivalents to the quantities. The term “about” can mean within 10% of the reported numerical value, preferably within 5% of the reported numerical value.

The term “therapeutic anticaries activity”, as used herein, is the anticaries activity provided by a composition including an anticaries drug and/or one or more anticaries agents in a therapeutic dose. A therapeutic dose for fluoride ions is defined in the United States by the Food and Drug Administration (FDA) Monograph (21 CFR part 355). For example, a therapeutic amount of sodium fluoride in a paste dosage form (i.e. paste dentifrice) is 850 to 1,150 ppm with an available fluoride ion concentration of at least 650 ppm. Other anticaries drugs are disclosed in 21 CFR part 355, which is herein incorporated by reference. Other therapeutic dosages are available in respective jurisdictions. Thus, as used herein, therapeutic anticaries activity for a composition comprising one or more anticaries agents is the anticaries benefit which is equivalent or better than the anticaries benefit provided by a composition comprising sodium fluoride with at least 650 ppm of available fluoride ions.

The term “subtherapeutic anticaries activity”, as used herein, is the anticaries activity of a composition that has a lower anticaries benefit relative to the anticaries benefit provided by a composition comprising sodium fluoride with at least 650 ppm of available fluoride ions, as defined in “therapeutic anticaries drug composition.” The term “subtherapeutic anticaries activity” also can also describe anticaries agents that can, in combination with additional anticaries agents, lead to a therapeutic benefit relative to the anticaries benefit provided by a composition comprising sodium fluoride with at least 650 ppm of available fluoride ions, when utilized in an oral care composition suitable for use in the oral cavity of a human, but alone has not been shown to provide a therapeutic benefit at concentrations suitable for use in an oral care composition.

The term “anticaries drug”, as used herein, is a drug that aids in the prevention and prophylactic treatment of dental cavities (decay, caries). This can include fluoride ion sources, such as in 21 CFR part 355 and anticaries agents, as described herein.

“Array” means a display of packages comprising oral care compositions comprising varying amounts and identities of anticaries drugs. The packages may have the same brand and/or sub-brand and/or the same trademark registration and/or having been manufactured by or for a common manufacturer and the packages may be available at a common point of sale (e.g. oriented in proximity to each other in a given area of a retail store or organized together on the same website). An array is marketed as a line-up of products normally having like packaging elements (e.g., packaging material type, film, paper, dominant color, design theme, etc.) that convey to consumers that the different individual packages are part of a larger line-up. Arrays often have the same brand, for example, “Crest,” and same sub-brand, for example, “Pro-Health.” A different product in the array may have the same brand “Crest” and, optionally a different sub-brand “3D White.” The differences between the “Pro-Health” product of the array and the “3D White” product in the array may include product form, different anticaries drug, different amounts of the anticaries drug, or other differences in other active or inactive ingredients. Arrays also often have the same trademarks, including trademarks of the brand, sub-brand, and/or features and/or benefits across the line-up. “On-line Array” means an “Array” distributed by a common on-line source.

The oral care compositions that make up the array can be in any suitable form, such as a solid, liquid, powder, paste, or combinations thereof. The oral care composition can be dentifrice, tooth gel, subgingival gel, mouth rinse, mousse, foam, mouth spray, lozenge, chewable tablet, chewing gum, tooth whitening strips, floss and floss coatings, breath freshening dissolvable strips, or denture care or adhesive product. The components of a dentifrice composition can be incorporated into a film, a strip, a foam, or a fiber-based dentifrice composition. The oral care compositions can include a variety of active and inactive ingredients, such as, for example, but not limited to a hops extract, a tin ion source, a calcium ion source, water, a fluoride ion source, zinc ion source, one or more polyphosphates, humectants, surfactants, other ingredients, and the like, as well as any combination thereof, as described below.

Section headers are provided below for organization and convenience only. The section headers do not suggest that a compound cannot be within more than one section. In fact, compounds can fall within more than one section. For example, stannous chloride can be both a tin ion source and a biofilm modifier, stannous fluoride can be both a tin ion source and a fluoride ion source, glycine can be an amino acid, a buffering agent, and/or a biofilm modifier, among numerous other compounds that can fit amongst several categories and/or sections.

### Arrays

The arrays, as described herein, comprise at least two oral care compositions. The oral care compositions that make up the array can be commonly marketed in the same line-up, under the same primary brand or trademark, available on shelf in a common location within a store, available from the same website with the same web domain, such as for example “www.crest.com”, and/or listed by the same seller on a third party website, such as Amazon, Alibaba, JD, and/or Rakuten.

The array can comprise dentifrice compositions, toothpaste compositions, tooth gel compositions, subgingival gel compositions, mouth rinse compositions, mousse compositions, foam compositions, mouth spray compositions, lozenge compositions, chewable tablet compositions, chewing gum compositions, tooth whitening strips, floss and floss coatings, breath freshening dissolvable strips, denture care compositions, and/or combinations thereof. The oral care composition may also be incorporated onto strips or films for direct application or attachment to oral surfaces.

The arrays, as described herein, can comprise at least two oral care compositions with either varying fluoride amount and/or varying anticaries activity. The anticaries activity can be affected by adding anticaries agents other than fluoride, replacing fluoride with other anticaries agents, or adding anticaries agents to compositions already comprising fluoride.

The array can comprise two or more, three or more, and/or four or more of the oral care compositions described below. The oral care compositions described below can be anticavity oral care compositions. The array can comprise a Category A composition, a Category B composition, a Category C composition and/or a Category D composition.

Category A Compositions

The array can comprise an oral care composition comprising a therapeutic dose of fluoride (Category A compositions) as described herein. Category A compositions can also include other oral care components, as described herein. Suitable other oral care components include tin, zinc, calcium, abrasive, polyphosphate, buffering agent, biofilm modifier, amino acid, and/or combinations thereof.

Category B Compositions

The array can comprise an oral care composition comprising a therapeutic dose of fluoride and additional anticaries agent (Category B compositions). The anticaries activity of Category B compositions can be greater than the anticaries activity of Category A compositions. Category B compositions can also include other oral care components, as described herein. Suitable other oral care components include tin, zinc, calcium, abrasive, polyphosphate, buffering agent, biofilm modifier, amino acid, and/or combinations thereof.

Category C Compositions

The array can comprise an oral care composition comprising a subtherapeutic dose of fluoride and additional subtherapeutic anticaries agent (Category C composition). The anticaries activity of a Category C composition can collectively provide a therapeutic benefit and can be at least about the anticaries activity of a Category A composition. The Category C compositions can also include other oral care components, as described herein. Suitable other oral care components include tin, zinc, calcium, abrasive, polyphosphate, buffering agent, biofilm modifier, amino acid, and/or combinations thereof.

Category D Composition

The array can comprise an oral care composition comprising a first subtherapeutic anticaries agent and a second subtherapeutic anticaries agent, but wherein the oral care composition is free of fluoride (Category D composition). The Category D compositions can collectively provide a therapeutic anticaries benefit and can be at least about the anticaries activity of a Category A composition. The Category D compositions can also include other oral care components, as described herein. Suitable other oral care components include tin, zinc, calcium, abrasive, polyphosphate, buffering agent, biofilm modifier, amino acid, and/or combinations thereof.

Suitable oral care compositions that make up the array are described in TABLE 1. The array can comprise two or more, three or more, and/or four or more of the oral care compositions described in TABLE 1 and/or TABLE 2.

TABLE 1
Suitable Oral Care Compositions that can be included in the Array

| | Composition | Composition | Composition | Composition |
|---|---|---|---|---|
| | 1 | 2 | 3 | 4 |
| Anticaries | Therapeutic | Therapeutic | Sub- | First Sub- |
| Active | Amount of | Amount of | therapeutic | therapeutic |
| Agent 1 | Fluoride | Fluoride | Dose of | Anticaries |
| | | | Fluoride | Agent |
| Anticaries | None | Sub- | Sub- | Second Sub- |
| Active | | therapeutic | therapeutic | therapeutic |
| Agent 2 | | Anticaries | Anticaries | Anticaries |
| | | Agent | Agent | Agent |
| Amount of | Therapeutic | Therapeutic | Sub- | Free of |
| Fluoride | Amount | Amount | therapeutic | Fluoride |
| | | | Amount | |
| Anticaries | Therapeutic | Greater than | Therapeutic | Therapeutic |
| Activity of | Anticaries | Therapeutic | Anticaries | Anticaries |
| Composition | Anticavity | Anticaries | Activity | Activity |
| | | Activity | | |
| Category | A | B | C | D |

TABLE 1 four possible compositions of the array of the present invention. Composition 1 is a fluoride anticavity oral care composition. Composition 1 includes a therapeutic amount of fluoride as required by the relevant regulatory agency in a jurisdiction of interest, such as the U.S. FDA in the United States. As previously described, many consumers have the perception that fluoride should be avoided. This has led to the increase in the use of fluoride free compositions, which likely do not include other anticaries agents, and thus no anticaries activity. Thus, many consumers desire alternatives to fluoride, such as Composition 3 (low fluoride) and Composition 4 (no fluoride) both of which have a therapeutic anticavity benefit in total by replacing all or some of the fluoride with one or more alternative anticaries agents. This will give consumers the choice of the amount of fluoride in the oral care composition while still providing an anticaries benefit.

Additionally, TABLE 1 describes Composition 2 which comprises a therapeutic amount of fluoride and an additional anticaries agent, which provide a greater than a therapeutic anticavity benefit. In many jurisdictions, a composition with a greater than therapeutic dose of fluoride will require is not available over-the-counter (i.e. without the administration by or a prescription from a medical professional, such as a dentist). Thus, Composition 2 can provide a superior anticaries benefit without having to increase the amount of fluoride in the composition.

TABLE 2
Possible Oral Care Compositions that can be included in the Array

| | Composition | Composition | Composition | Composition |
|---|---|---|---|---|
| | 5 | 6 | 7 | 8 |
| Anticaries | Stannous | Stannous | Stannous | Stannous |
| Agent 1 | Fluoride | Fluoride | Fluoride | Chloride |
| Amount | 0.454 wt % | 0.454 wt % | Sub- | Sub- |
| | (therapeutic) | (therapeutic) | therapeutic | therapeutic |
| Anticaries | N/A | Hops | Hops | Hops |
| Agent 2 | | | | |
| Amount | N/A | Sub- | Sub- | Sub- |
| | | therapeutic | therapeutic | therapeutic |
| Anticaries | Therapeutic | Greater than | Therapeutic | Therapeutic |
| Activity of | | Therapeutic | | |
| Composition | | | | |
| Category | A | B | C | D |

For example, as shown in TABLE 2, Composition 5 can include stannous fluoride in a therapeutic dose to give a therapeutic anticaries activity. Composition 6 can provide greater than therapeutic anticaries activity by combining a therapeutic dose of stannous fluoride with a subtherapeutic anticaries agent, such as hops. Composition 7 can provide therapeutic anticaries activity therapeutic activity by combining a subtherapeutic dose of stannous fluoride with a subtherapeutic anticaries agent, such as hops. Composition 8 can provide therapeutic anticaries activity therapeutic activity by combining a first subtherapeutic anticaries agent, such as stannous chloride, with a second subtherapeutic anticaries agent, such as hops. In each composition in TABLE 2, the anticaries agent(s) can be combined with other oral care components, as described herein. Suitable other oral care components include tin, zinc, calcium, abrasive, polyphosphate, buffering agent, biofilm modifier, amino acid, and/or combinations thereof. Additionally, other fluoride-containing compounds can be used in place of stannous fluoride, such as sodium fluoride, sodium monofluorophosphate, and/or amine fluoride, as described herein.

An array comprising a composition from Category A and a composition from Category B can give a consumer the ability to pick from two over-the-counter compositions, one with normal anticaries activity (Category A) and one with superior anticaries activity (Category B), without having to obtain a prescription or having to get it applied by a medical professional, such as a dentist or dental hygienist.

An array comprising at least two of a composition from Category A (therapeutic amount of fluoride), a composition from Category C (subtherapeutic dose of fluoride), and a composition from Category D (no fluoride) can give a consumer the choice in the amount of fluoride without sacrificing anticaries activity.

### Anticaries Activity

The oral care compositions, as described herein, can comprise one or more anticaries agents, which collectively or individually demonstrate therapeutic anticaries activity. As described herein, therapeutic anticaries activity is defined by the relevant regulatory agency in a jurisdiction of interest, such as the U.S. FDA in the United States. At the FDA, a therapeutic amount of sodium fluoride in a paste dosage form (i.e. paste dentifrice) is 850 to 1,150 ppm with an available fluoride ion concentration of at least 650 ppm. Thus, the oral care compositions of the present invention can have an anticaries benefit at least about of the anticaries benefit of a composition comprising at least, at least about, or greater than 650 ppm, 800 ppm, 850 ppm, 1100 ppm, 1150 ppm, 1450 ppm, and/or 2800 ppm of free fluoride ions, which can correspond to therapeutic anticaries activity.

The anticaries activity of the oral care compositions, as described herein, can also be described by a rat caries score. The oral care compositions can have a rat caries score of about 35 or less, about 30 or less, about 25 or less, and/or about 20 or less. The anticaries activity of the oral care compositions, as described herein, can also be described the percent reduction of the rat caries score relative to a placebo toothpaste. The oral care compositions can have a percent reduction in caries relative to a placebo toothpaste (i.e. a negative control) of at least about 25%, at least about 29%, at least about 30%, at least about 35%, and/or at least about 40%.

The anticaries activity of the oral care compositions, as described herein, can also be described by a percent reduction of the rat caries score relative to an oral care composition comprising a therapeutic dose of an anticaries agent (i.e. a positive control, such as USP NaF). The oral care compositions can have a percent reduction in caries relative to a positive control, such as USP NaF, of about 50% or more, about 60% or more, about 65% or more, about 70% or more, about 75% or more, about 80% or more, about 90% or more, about 100% or more, about 125% or more, and/or about 150% or more.

### Anticaries Agent

The oral care composition, as described herein, comprise one or more anticaries compositions, the one or more anticaries compositions comprising one or more anticaries agents, which can, individually, be in a therapeutic dose or a subtherapeutic dose.

The oral care composition can comprise a first subtherapeutic anticaries agent and a second subtherapeutic anticaries agent, which in total lead to a therapeutic anticaries benefit. The first and second subtherapeutic anti caries agents can be fluoride-free, as described herein, or one of the subtherapeutic agents can comprise a subtherapeutic amount of fluoride ions.

The oral care composition can comprise a therapeutic anticaries agent comprising a fluoride ion source and a subtherapeutic anticaries agent, which is free from a fluoride ion source, where the overall oral care composition has a higher anticaries benefit than the anticaries benefit associated with the a therapeutic anticaries composition alone. The anticaries activity of a composition comprising a therapeutic anticaries agent and a subtherapeutic anticaries agent can allow for anticaries activity that is normally available with a prescription strength concentration of fluoride ions.

The anticaries agent can be active against caries through one of these four mechanisms: i) suppressing acid formation via antibacterial action; ii) reducing enamel solubility through a calcium co-ion effect; iii) reducing enamel solubility through a fluoride co-ion effect; and iv) reducing enamel solubility through surface adsorbed stabilizers. Thus, the anticaries agent can be an antibacterial agent, a calcium ion source, a fluoride ion source, a surface adsorbed stabilizer. However, a compound can fall within more than one of these categories, such as, for example, stannous chloride, which can be an antibacterial agent and a surface adsorbed stabilizer or stannous fluoride, which can be an antibacterial agent, a fluoride ion source, and a surface adsorbed stabilizer.

### Antibacterial Agent

The oral care composition can comprise one or more anticaries agents, the one or more anticaries agents can comprise one or more antibacterial agents. The antibacterial agent can be any agent that suppresses acid formation by the bacteria of dental caries. Suitable antibacterial agents include agents that those that can provide at least about an 80%, or about 30%, 60%, 65%, 75%, 85%, 90%, or 95%, reduction in ApH with respect to Crest® Cavity Protection that thereby reduce caries at least about 9%, or about 1%, 6%, 7%, 8%, 10%, 11%, or 12%, with respect to the placebo or water control in rat caries experiments.

Suitable antibacterial agents include hops, hops acids, such as hops alpha acids, hops beta acids, hydrogenated hops acids, and/or combinations thereof. Other suitable antibacterial agents include metal ion sources, such as tin ion sources, zinc ion sources, copper ion sources, and/or combinations thereof. Other suitable antibacterial agents include triclosan, extracts from any species within the genus Magnolia, extracts from any species within the genus Humulus. Other suitable antibacterial agents include hops acids, tin ion sources, benzyl alcohol, sodium benzoate, menthylglycyl acetate, menthyl lactate, L-menthol, o-neomenthol, chlorophyllin copper complex, phenol, oxyquinoline, and/or combinations thereof. Other suitable antibacterial agents include one or more amino acids, such as basic amino acids.

The oral care composition can comprise from about 0.01% to about 10%, from about 1% to about 5%, or from about 0.5% to about 15% of an antibacterial agent. Some, but not all, suitable antibacterial agents will be discussed separately.

### Surface Adsorbed Stabilizers

The oral care composition can comprise one or more anticaries agents, the one or more anticaries agents can comprise one or more surface adsorbed stabilizers. The surface adsorbed stabilizer can be any agent that can adsorb on an enamel surface. A suitable surface adsorbed stabilizer can be any compound that can provide at least a 17%, at least a 5%, or at least a 20%, at least a 30%, and/or at least a 15%, reduction in solubility relative to water in the F-free HAP dissolution method, which thereby reduces caries at least 5%, 6%, 7%, 8%, 9%, 10%, 11%, 12%, 13%, and/or 18% with respect to the placebo or water control in rat caries experiments.

Suitable surface adsorbed stabilizers include metal ion sources, such as tin ion sources, zinc ion sources, copper ion sources, aluminum ion sources, titanium ion sources and/or combinations thereof. Other suitable surface adsorbed stabilizers include bioactive materials, amino acids, and/or combinations thereof. Some, but not all, suitable surface adsorbed stabilizes will be discussed separately.

Humulus lupulus

The oral care compositions of the present invention can comprise hops, such as at least one hops compound from Formula I and/or Formula IV. The compound from Formula I and/or Formula IV can be provided by any suitable source, such as an extract from Humulus lupulus or Hops, Humulus lupulus itself, a synthetically derived compound, and/or salts, prodrugs, or other analogs thereof. The hops extract can comprise one or more hops alpha acids, one or more hops iso-alpha acids, one or more hops beta acids, one or more hops oils, one or more flavonoids, one or more solvents, and/or water. Suitable hops alpha acids (generically shown in Formula I) can include humulone (Formula II), adhumulone, cohumulone, posthumulone, prehumulone, and/or mixtures thereof. Suitable hops iso-alpha acids can include cis-isohumulone and/or trans-isohumulone. The isomerization of humulone into cis-isohumulone and trans-isohumulone can be represented by Formula III.

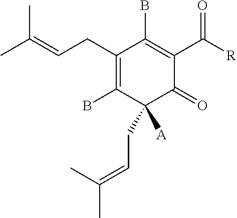

Formula I. Hops Alpha Acids. A is the acidic hydroxyl functional group in the alpha position, B are the acidic hydroxyl functional groups in the beta position, and R is an alkyl functional group.

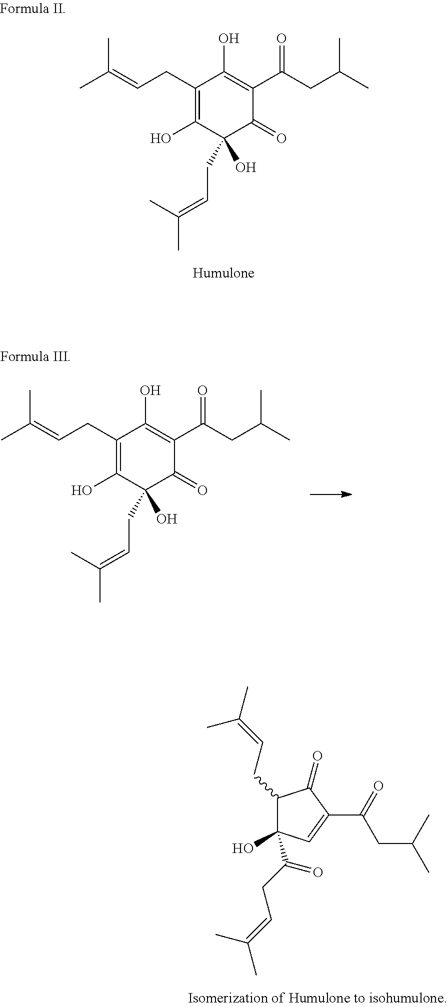

Suitable hops beta acids can include lupulone, adlupulone, colupulone, and/or mixtures thereof. A suitable hops beta acid can include a compound a described in Formula IV, V, VI, and/or VII.

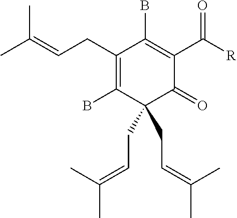

Formula IV. Hops Beta Acids. B are the acidic hydroxyl functional groups in the beta position and R is an alkyl functional group.

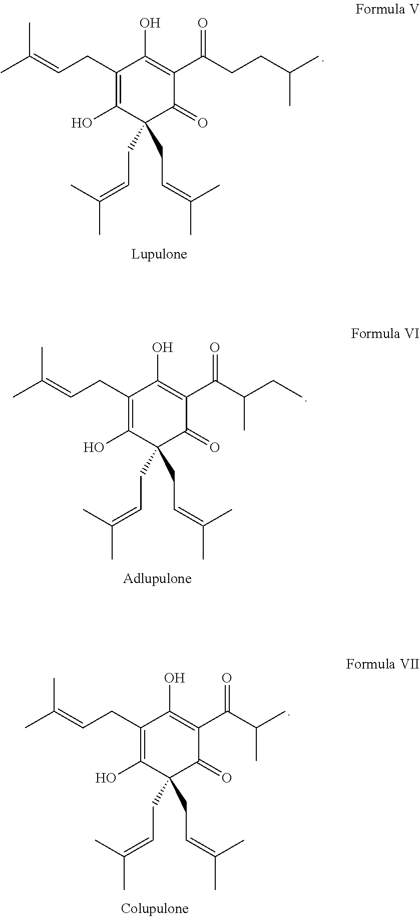

While hops alpha acids can demonstrate some antibacterial activity, hops alpha acids also have a bitter taste. The bitterness provided by hops alpha acids can be suitable for beer, but are not suitable for use in oral care compositions. In contrast, hops beta acids can be associated with a higher antibacterial and/or anticaries activity, but not as bitter a taste. Thus, a hops extract with a higher proportion of beta acids to alpha acids than normally found in nature, can be suitable for use in oral care compositions for use as an antibacterial and/or anticaries agent.

A natural hops source can comprise from about 2% to about 12%, by weight of the hops source, of hops beta acids depending on the variety of hops. Hops extracts used in other contexts, such as in the brewing of beer, can comprise from about 15% to about 35%, by weight of the extract, of hops beta acids. The hops extract desired herein can comprise at least about 35%, at least about 40%, at least about 45%, from about 35% to about 95%, from about 40% to about 90%, or from about 45% to about 99%, of hops beta acids. The hops beta acids can be in an acidic form (i.e. with attached hydrogen atom(s) to the hydroxl functional group(s)) or as a salt form.

A suitable hops extract is described in detail in U.S. Pat. No. 7,910,140, which is herein incorporated by reference in its entirety. The hops beta acids desired can be non-hydrogenated, partially hydrogenated by a non-naturally occurring chemical reaction, or hydrogenated by a non-naturally occurring chemical reaction. The hops beta acid can be essentially free of or substantially free of hydrogenated hops beta acid and/or hops acid. A non-naturally occurring chemical reaction is a chemical reaction that was conducted with the aid of chemical compound not found within Humulus lupulus, such as a chemical hydrogenation reaction conducted with high heat not normally experienced by Humulus lupulus in the wild and/or a metal catalyst.

A natural hops source can comprise from about 2% to about 12%, by weight of the hops source, of hops alpha acids. Hops extracts used in other contexts, such as in the brewing of beer, can comprise from about 15% to about 35%, by weight of the extract, of hops alpha acids. The hops extract desired herein can comprise less than about 10%, less than about 5%, less than about 1%, or less than about 0.5%, by weight of the extract, of hops alpha acids.

Hops oils can include terpene hydrocarbons, such as myrcene, humulene, caryophyllene, and/or mixtures thereof. The hops extract desired herein can comprise less than 5%, less than 2.5%, or less than 2%, by weight of the extract, of one or more hops oils.

Flavonoids present in the hops extract can include xanthohumol, 8-prenylnaringenin, isoxanthohumol, and/or mixtures thereof. The hops extract can be substantially free of, essentially free of, free of, or have less than 250 ppm, less than 150 ppm, and/or less than 100 ppm of one or more flavonoids.

As described in U.S. Pat. No. 5,370,863, hops acids have been previously added to oral care compositions. However, the oral care compositions taught by U.S. Pat. No. 5,370,863 only included up to 0.01%, by weight of the oral care composition. While not wishing to be bound by theory, it is believed that U.S. Pat. No. 5,370,863 could only incorporate a low amount of hops acids because of the bitterness of hops alpha acids. A hops extract with a low level of hops alpha acids would not have this concern.

The hops compound can be combined with or free from an extract from another plant, such as a species from genus Magnolia. The hops compounds can be combined with or free from triclosan.

The oral care composition can comprise from about 0.01% to about 10%, greater than 0.01% to about 10%, from about 0.05%, to about 10%, from about 0.1% to about 10%, from about 0.2% to about 10%, from about 0.2% to about 10%, from about 0.2% to about 5%, from about 0.25% to about 2%, from about 0.05% to about 2%, or from greater than 0.25% to about 2%, of hops, such as hops beta acid, as described herein. The hops, such as the hops beta acid, can be provided by a suitable hops extract, the hops plant itself, or a synthetically derived compound. The hops, such as hops beta acid, can be provided as neutral, acidic compounds, and/or as salts with a suitable counter ion, such as sodium, potassium, ammonia, or any other suitable counter ion.

The hops can be provided by a hops extract, such as an extract from Humulus lupulus with at least 35%, by weight of the extract, of hops beta acid and less than 1%, by weight of the hops extract, of hops alpha acid. The oral care composition can comprise 0.01% to about 10%, greater than 0.01% to about 10%, from about 0.05%, to about 10%, from about 0.1% to about 10%, from about 0.2% to about 10%, from about 0.2% to about 10%, from about 0.2% to about 5%, from about 0.25% to about 2%, from about 0.05% to about 2%, or from greater than 0.25% to about 2%, of hops extract, as described herein.

### Fluoride Ion Source

The oral care composition can comprise fluoride, such as from a fluoride ion source. The fluoride ion source can comprise one or more fluoride containing compounds, such as stannous fluoride, sodium fluoride, titanium fluoride, calcium fluoride, calcium phosphate silicate fluoride potassium fluoride, amine fluoride, sodium monofluorophosphate, zinc fluoride, and/or mixtures thereof.

The fluoride ion source and the tin ion source can be the same compound, such as for example, stannous fluoride, which can generate tin ions and fluoride ions. Additionally, the fluoride ion source and the tin ion source can be separate compounds, such as when the tin ion source is stannous chloride and the fluoride ion source is sodium monofluorophosphate or sodium fluoride.

The fluoride ion source and the zinc ion source can be the same compound, such as for example, zinc fluoride, which can generate zinc ions and fluoride ions. Additionally, the fluoride ion source and the zinc ion source can be separate compounds, such as when the zinc ion source is zinc phosphate and the fluoride ion source is stannous fluoride.

The fluoride ion source can be essentially free of or free of stannous fluoride. Thus, the oral care composition can comprise sodium fluoride, potassium fluoride, amine fluoride, sodium monofluorophosphate, zinc fluoride, and/or mixtures thereof.

The oral care composition can comprise a fluoride ion source capable of providing from about 50 ppm to about 5000 ppm, and preferably from about 500 ppm to about 3000 ppm of free fluoride ions. To deliver the desired amount of fluoride ions, the fluoride ion source may be present in the oral care composition at an amount of from about 0.0025% to about 5%, from about 0.01% to about 10%, from about 0.2% to about 1%, from about 0.5% to about 1.5%, or from about 0.3% to about 0.6%, by weight of the oral care composition. Alternatively, the oral care composition can comprise less than 0.1%, less than 0.01%, be essentially free of, substantially free of, or free of a fluoride ion source.

### Tin Ion Source

The oral care composition of the present invention can comprise tin, such as from a tin ion source. The tin ion source can be any suitable compound that can provide tin ions in an oral care composition and/or deliver tin ions to the oral cavity when the dentifrice composition is applied to the oral cavity. The tin ion source can comprise one or more tin containing compounds, such as stannous fluoride, stannous chloride, stannous bromide, stannous iodide, stannous oxide, stannous oxalate, stannous sulfate, stannous sulfide, stannic fluoride, stannic chloride, stannic bromide, stannic iodide, stannic sulfide, and/or mixtures thereof. Tin ion source can comprise stannous fluoride, stannous chloride, and/or mixture thereof. The tin ion source can also be a fluoride-free tin ion source, such as stannous chloride.

The oral care composition can comprise from about 0.0025% to about 5%, from about 0.01% to about 10%, from about 0.2% to about 1%, from about 0.5% to about 1.5%, or from about 0.3% to about 0.6%, by weight of the oral care composition, of a tin ion source.

### Ca Ion Source

The oral care composition of the present invention can comprise calcium, such as from a calcium ion source. The calcium ion source can be any suitable compound or molecule that can provide calcium ions in an oral care composition and/or deliver calcium ions to the oral cavity when the oral care composition is applied to the oral cavity. The calcium ion source can comprise a calcium salt, a calcium abrasive, and/or combinations thereof. In some cases, a calcium salt may also be considered a calcium abrasive or a calcium abrasive may also be considered a calcium salt.

The calcium ion source can comprise a calcium abrasive. The calcium abrasive can be any suitable abrasive compound that can provide calcium ions in an oral care composition and/or deliver calcium ions to the oral cavity when the oral care composition is applied to the oral cavity. The calcium abrasive can comprise one or more calcium abrasive compounds, such as calcium carbonate, precipitated calcium carbonate (PCC), ground calcium carbonate (GCC), chalk, dicalcium phosphate, calcium pyrophosphate, and/or mixtures thereof.

The calcium ion source can comprise a calcium salt, or a compound that can provide calcium ions in an oral care composition and/or deliver calcium ions to the oral cavity when the oral care composition is applied to the oral cavity that can not act as an abrasive. The calcium salt can comprise one or more calcium compounds, such as calcium chloride, calcium nitrate, calcium phosphate, calcium lactate, calcium oxalate, calcium oxide, calcium gluconate, calcium citrate, calcium bromide, calcium iodate, calcium iodide, hydroxyapatite, fluorapatite, calcium sulfate, calcium glycerophosphate, and/or combinations thereof.

The oral care composition can comprise from about 5% to about 70%, from about 10% to about 50%, from about 10% to about 60%, from about 20% to about 50%, from about 25% to about 40%, or from about 1% to about 50% of a calcium ion source.

### Buffering Agent

The oral care composition can comprise a buffering agent. The buffering agent can be a weak acid or base that can maintain a particular pH at a selected site in the oral cavity. For example, the buffering agent can maintain a pH at a tooth's surface to mitigate the impact of plaque acids produced by bacteria. The buffering agent can comprise a conjugate acid of an ion also present in the oral care composition. For example, if the calcium ion source comprises calcium carbonate, the buffering agent can comprise a bicarbonate anion (—HCO₃⁻). The buffering agent can comprise a conjugate acid/base pair, such as citric acid and sodium citrate.

Suitable buffering systems can include phosphate, citrate salts, carbonate/bicarbonate salts, a tris buffer, imidazole, urea, borate, and/or combinations thereof. Suitable buffering agents include bicarbonate salts, such as sodium bicarbonate, glycine, orthophosphate, arginine, urea, and or/combinations thereof.

The oral care composition can comprise from about 1% to about 30%, from about 5% to about 25% or from about 10% to about 20%, of one or more buffering agents.

### Biofilm Modifier

The oral care composition can comprise one or more biofilm modifiers. A biofilm modifier can comprise a polyol, an ammonia generating compound, and/or a glucosyltransferase inhibitor.

A polyol is an organic compound with more than one hydroxyl functional groups. The polyol can be any suitable compound that can weakly associate, interact, or bond to tin ions while the oral care composition is stored prior to use. The polyol can be a sugar alcohol, which area class of polyols that can be obtained through the hydrogenation of sugar compounds with the formula (CHOH)ₙH₂. The polyol can be glycerin, erythritol, xylitol, sorbitol, mannitol, butylene glycol, lactitol, and/or combinations thereof. The oral care composition can comprise 0.01% to about 70%, from about 5% to about 70%, from about 5% to about 50%, from about 10% to about 60%, from about 10% to about 25%, or from about 20% to about 80%, by weight of the oral care composition, of a polyol.

The ammonia generating compound can be any suitable compound that can generate ammonia upon delivery to the oral cavity. Suitable ammonia generating compounds include arginine, urea, and/or combinations thereof. The oral care composition can comprise from about 0.01% to about 10%, from about 1% to about 5%, or from about 1% to about 25% of one or more ammonia generating compounds.

The glucosyltransferase inhibitor can be any suitable compound that can inhibit a glucosyltransferase. Glucosyltransferases are enzymes that can establish natural glycosidic linkages. In particular, these enzymes break down poly- or oligosaccharide moieties into simple sugars for bacteria associated with dental caries. As such, any compound that can inhibit this process can help prevent dental caries. Suitable glucosyltransferase inhibitors include oleic acid, epicatechin, tannins, tannic acid, moenomycin, caspofungin, ethambutol, lufenuron, and/or combinations thereof. The oral care composition can comprise from about 0.001% to about 5%, from about 0.01% to about 2%, or about 1% of one or more glucosyltransferase inhibitors.

### Metal Ion Source

The oral care composition can comprise metal, such as from a metal ion source comprising one or more metal ions. The metal ion source can comprise or be in addition to the tin ion source and/or the zinc ion source, as described herein. Suitable metal ion sources include compounds with metal ions, such as, but not limited to Sn, Zn, Cu, Mn, Mg, Sr, Ti, Fe, Mo, B, Ba, Ce, Al, In and/or mixtures thereof. The trace metal source can be any compound with a suitable metal and any accompanying ligands and/or anions.

Suitable ligands and/or anions that can be paired with metal ion sources include, but are not limited to acetate, ammonium sulfate, benzoate, bromide, borate, carbonate, chloride, citrate, gluconate, glycerophosphate, hydroxide, iodide, oxide, propionate, D-lactate, DL-lactate, orthophosphate, pyrophosphate, sulfate, nitrate, tartrate, and/or mixtures thereof.

The oral care composition can comprise from about 0.01% to about 10%, from about 1% to about 5%, or from about 0.5% to about 15% of a metal ion source.

### Antibacterial Agents

The oral care composition can comprise one or more antibacterial agents. Suitable antibacterial agents include any molecule that provides antibacterial activity in the oral cavity. Suitable antibacterial agents include hops acids, tin ion sources, benzyl alcohol, sodium benzoate, menthylglycyl acetate, menthyl lactate, L-menthol, o-neomenthol, chlorophyllin copper complex, phenol, oxyquinoline, and/or combinations thereof.

The oral care composition can comprise from about 0.01% to about 10%, from about 1% to about 5%, or from about 0.5% to about 15% of an antibacterial agent.

### Bioactive Materials

The oral care composition can also include bioactive materials suitable for the remineralization of a tooth. Suitable bioactive materials include bioactive glasses, Novamin™, Recaldent™ hydroxyapatite, one or more amino acids, such as, for example, arginine, citrulline, glycine, lysine, or histidine, or combinations thereof. Suitable examples of compositions comprising arginine are found in U.S. Pat. Nos. 4,154,813 and 5,762,911, which are herein incorporated by reference in their entirety. Other suitable bioactive materials include any calcium phosphate compound. Other suitable bioactive materials include compounds comprising a calcium source and a phosphate source.

Amino acids are organic compounds that contain an amine functional group, a carboxyl functional group, and a side chain specific to each amino acid. Suitable amino acids include, for example, amino acids with a positive or negative side chain, amino acids with an acidic or basic side chain, amino acids with polar uncharged side chains, amino acids with hydrophobic side chains, and/or combinations thereof. Suitable amino acids also include, for example, arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, citrulline, ornithine, creatine, diaminobutonic acid, diaminoproprionic acid, salts thereof, and/or combinations thereof.

Bioactive glasses are comprising calcium and/or phosphate which can be present in a proportion that is similar to hydroxyapatite. These glasses can bond to the tissue and are biocompatible. Bioactive glasses can include a phosphopeptide, a calcium source, phosphate source, a silica source, a sodium source, and/or combinations thereof.

The oral care composition can comprise from about 0.01% to about 20%, from about 0.1% to about 10%, or from about 1% to about 10% of a bioactive material by weight of the oral care composition.

### Abrasive

The oral care composition can comprise a calcium abrasive, as described herein, and/or a non-calcium abrasive, such as bentonite, silica gel (by itself, and of any structure), precipitated silica, amorphous precipitated silica (by itself, and of any structure as well), hydrated silica, perlite, titanium dioxide, calcium pyrophosphate, dicalcium phosphate dihydrate, alumina, hydrated alumina, calcined alumina, aluminum silicate, insoluble sodium metaphosphate, insoluble potassium metaphosphate, insoluble magnesium carbonate, zirconium silicate, particulate thermosetting resins and other suitable abrasive materials. Such materials can be introduced into the oral care compositions to tailor the polishing characteristics of the target dentifrice formulation. The oral care composition can comprise from about 5% to about 70%, from about 10% to about 50%, from about 10% to about 60%, from about 20% to about 50%, from about 25% to about 40%, or from about 1% to about 50%, by weight of the oral care composition, of the non-calcium abrasive.

Alternatively, the oral care composition can be substantially free of, essentially free of, or free of silica, alumina, or any other non-calcium abrasive. The oral care composition can comprise less than about 5%, less than about 1%, less than about 0.5%, less than about 0.1%, or 0% of a non-calcium abrasive, such as silica and/or alumina.

### Water

The oral care composition of the present invention can be anhydrous, a low water formulation, or a high water formulation. In total, the oral care composition can comprise from 0% to about 99%, from about 5% to about 75%, about 20% or greater, about 30% or greater, or about 50% or greater by weight of the composition, of water. Preferably, the water is USP water.

In a high water oral care composition and/or toothpaste formulation, the oral care composition comprises from about 45% to about 75%, by weight of the composition, of water. The high water oral care composition and/or toothpaste formulation can comprise from about 45% to about 65%, from about 45% to about 55%, or from about 46% to about 54%, by weight of the composition, of water. The water may be added to the high water formulation and/or may come into the composition from the inclusion of other ingredients.

In a low water oral care composition and/or toothpaste formulation, the oral care composition comprises from about 5% to about 45%, by weight of the composition, of water. The low water oral care composition can comprise from about 5% to about 35%, from about 10% to about 25%, or from about 20% to about 25%, by weight of the composition, of water. The water may be added to the low water formulation and/or may come into the composition from the inclusion of other ingredients.

In an anhydrous oral care composition and/or toothpaste formulation, the oral care composition comprises less than about 10%, by weight of the composition, of water. The anhydrous composition comprises less than about 5%, less than about 1%, or 0%, by weight of the composition, of water. The water may be added to the anhydrous formulation and/or may come into the composition from the inclusion of other ingredients.

A mouth rinse formulation comprises from about 75% to about 99%, from about 75% to about 95%, or from about 80% to about 95% of water.

The composition can also comprise other orally acceptable carrier materials, such as alcohol, humectants, polymers, surfactants, and acceptance improving agents, such as flavoring, sweetening, coloring and/or cooling agents.

pH

The pH of the disclosed composition can be from about 4 to about 10, from about 7 to about 10, greater than 7 to about 10, greater than 8 to about 10, greater than 7, greater than 7.5, greater than 8, greater than 9, or from about 8.5 to about 10.

### Zinc Ion Source

The oral care composition can comprise zinc, such as from a zinc ion source. The zinc ion source can comprise one or more zinc containing compounds, such as zinc fluoride, zinc lactate, zinc oxide, zinc phosphate, zinc chloride, zinc acetate, zinc hexafluorozirconate, zinc sulfate, zinc tartrate, zinc gluconate, zinc citrate, zinc malate, zinc glycinate, zinc pyrophosphate, zinc metaphosphate, zinc oxalate, and/or zinc carbonate. The zinc ion source can be a fluoride-free zinc ion source, such as zinc phosphate, zinc oxide, and/or zinc citrate.

The zinc ion source may be present in the total oral care composition at an amount of from about 0.01% to about 10%, from about 0.2% to about 1%, from about 0.5% to about 1.5%, or from about 0.3% to about 0.6%, by weight of the dentifrice composition.

### Polyphosphates

The oral care composition can comprise polyphosphate, such as from a polyphosphate source. A polyphosphate source can comprise one or more polyphosphate molecules. Polyphosphates are a class of materials obtained by the dehydration and condensation of orthophosphate to yield linear and cyclic polyphosphates of varying chain lengths. Thus, polyphosphate molecules are generally identified with an average number (n) of polyphosphate molecules, as described below. A polyphosphate is generally understood to consist of two or more phosphate molecules arranged primarily in a linear configuration, although some cyclic derivatives may be present.

Preferred polyphosphates are those having an average of two or more phosphate groups so that surface adsorption at effective concentrations produces sufficient non-bound phosphate functions, which enhance the anionic surface charge as well as hydrophilic character of the surfaces. Preferred in this invention are the linear polyphosphates having the formula: XO(XPO₃)ₙX, wherein X is sodium, potassium, ammonium, or any other alkali metal cations and n averages from about 2 to about 21. Alkali earth metal cations, such as calcium, are not preferred because they tend to form insoluble fluoride salts from aqueous solutions comprising a fluoride ions and alkali earth metal cations. Thus, the oral care compositions disclosed herein can be free of, essentially free of, or substantially free of calcium pyrophosphate.

Some examples of suitable polyphosphate molecules include, for example, pyrophosphate (n=2), tripolyphosphate (n=3), tetrapolyphosphate (n=4), sodaphos polyphosphate (n=6), hexaphos polyphosphate (n=13), benephos polyphosphate (n=14), hexametaphosphate (n=21), which is also known as Glass H. Polyphosphates can include those polyphosphate compounds manufactured by FMC Corporation, ICL Performance Products, and/or Astaris.

The oral care composition can comprise from about 0.01% to about 15%, from about 0.1% to about 10%, from about 0.5% to about 5%, from about 1 to about 20%, or about 10% or less, by weight of the oral care composition, of the polyphosphate source.

### Humectants

The oral care composition can comprise one or more humectants, have low levels of a humectant, be essentially free of, be substantially free of, or be free of a humectant. Humectants serve to add body or “mouth texture” to an oral care composition or dentifrice as well as preventing the dentifrice from drying out. Suitable humectants include polyethylene glycol (at a variety of different molecular weights), propylene glycol, glycerin (glycerol), erythritol, xylitol, sorbitol, mannitol, butylene glycol, lactitol, hydrogenated starch hydrolysates, and/or mixtures thereof. The oral care composition can comprise one or more humectants each at a level of from 0 to about 70%, from about 5% to about 50%, from about 10% to about 60%, or from about 20% to about 80%, by weight of the oral care composition.

### Surfactants

The oral care composition can comprise one or more surfactants. The surfactants can be used to make the compositions more cosmetically acceptable. The surfactant is preferably a detersive material which imparts to the composition detersive and foaming properties. Suitable surfactants are safe and effective amounts of anionic, cationic, nonionic, zwitterionic, amphoteric and betaine surfactants.

Suitable anionic surfactants include, for example, the water soluble salts of alkyl sulfates having from 8 to 20 carbon atoms in the alkyl radical and the water-soluble salts of sulfonated monoglycerides of fatty acids having from 8 to 20 carbon atoms. Sodium lauryl sulfate (SLS) and sodium coconut monoglyceride sulfonates are examples of anionic surfactants of this type. Other suitable anionic surfactants include sarcosinates, such as sodium lauroyl sarcosinate, taurates, sodium lauryl sulfoacetate, sodium lauroyl isethionate, sodium laureth carboxylate, and sodium dodecyl benzene sulfonate. Combinations of anionic surfactants can also be employed.

Another suitable class of anionic surfactants are alkyl phosphates. The surface active organophosphate agents can have a strong affinity for enamel surface and have sufficient surface binding propensity to desorb pellicle proteins and remain affixed to enamel surfaces. Suitable examples of organophosphate compounds include mono-, di- or triesters represented by the general structure below wherein Z₁, Z₂, or Z₃ may be identical or different with at least one being an organic moiety. Z₁, Z₂, or Z₃ can be selected from linear or branched, alkyl or alkenyl group of from 1 to 22 carbon atoms, optionally substituted by one or more phosphate groups; alkoxylated alkyl or alkenyl, (poly)saccharide, polyol or polyether group.

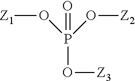

Some other agents include alkyl or alkenyl phosphate esters represented by the following structure:

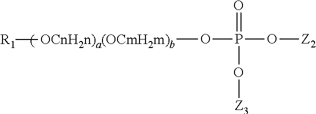

wherein R₁ represents a linear or branched, alkyl or alkenyl group of from 6 to 22 carbon atoms, optionally substituted by one or more phosphate groups; n and m, are individually and separately, 2 to 4, and a and b, individually and separately, are 0 to 20; Z and Z may be identical or different, each represents hydrogen, alkali metal, ammonium, protonated alkyl amine or protonated functional alkylamine, such as analkanolamine, or a R—(OCH₂)(OCH)— group. Examples of suitable agents include alkyl and alkyl (poly)alkoxy phosphates such as lauryl phosphate; PPGS ceteareth-10 phosphate; laureth-1 phosphate; laureth-3 phosphate; laureth-9 phosphate; trilaureth-4 phosphate; C₁₂₋₁₈ PEG 9 phosphate: and sodium dilaureth-10 phosphate. The alkyl phosphate can be polymeric. Examples of polymeric alkyl phosphates include those containing repeating alkoxy groups as the polymeric portion, in particular 3 or more ethoxy, propoxy isopropoxy or butoxy groups.

Other suitable anionic surfactants are sarcosinates, isethionates and taurates, especially their alkali metal or ammonium salts. Examples include: lauroyl sarcosinate, myristoyl sarcosinate, palmitoyl sarcosinate, stearoyl sarcosinate oleoyl sarcosinate, or combinations thereof.

Other suitable anionic surfactants include sodium or potassium alkyl sulfates, such as sodium lauryl sulfate, acyl isethionates, acyl methyl isethionates, alkyl ether carboxylates, acyl alaninates, acyl gulatames, acyl glycinates, acyl sarconsinates, sodium methyl acyl taurates, sodium laureth sulfosuccinates, alpha olefin sulfonates, alkyl benze sulfonates, sodium lauroyl lactylate, sodium laurylglucosides hydroxypropyl sulfonate, and/or combinations.

Zwitterionic or amphoteric surfactants useful herein include derivatives of aliphatic quaternary ammonium, phosphonium, and Sulfonium compounds, in which the aliphatic radicals can be straight chain or branched, and one of the aliphatic substituents contains from 8 to 18 carbon atoms and one contains an anionic water-solubilizing group, e.g., carboxy, sulfonate, sulfate, phosphate or phosphonate. Suitable betaine surfactants are disclosed in U.S. Pat. No. 5,180,577. Typical alkyl dimethyl betaines include decyl betaine or 2-(N-decyl-N,N-dimethylammonio) acetate, coco-betaine or 2-(N-coco-N,N-dimethyl ammonio)acetate, myristyl betaine, palmityl betaine, lauryl betaine, cetyl betaine, cetyl betaine, stearyl betaine, etc. The amidobetaines can be exemplified by cocoamidoethyl betaine, cocoamidopropyl betaine (CADB), and lauramidopropyl betaine. Other suitable amphoteric surfactants include betaines, sultaines, sodium laurylamphoacetates, alkylamphodiacetates, and/or combinations thereof.

Cationic surfactants useful in the present invention include, for example, derivatives of quaternary ammonium compounds having one long alkyl chain containing from 8 to 18 carbon atoms such as lauryl trimethylammonium chloride; cetyl pyridinium chloride; cetyl trimethyl-ammonium bromide; cetyl pyridinium fluoride or combinations thereof.

Nonionic surfactants that can be used in the compositions of the present invention include, for example, compounds produced by the condensation of alkylene oxide groups (hydrophilic in nature) with an organic hydrophobic compound which may be aliphatic or alkylaromatic in nature. Examples of suitable nonionic surfactants can include the Pluronics® which are poloxamers, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and combinations of such materials. Other suitable non-ionic surfactants includes alkyl glucamides, alkyl glucosides, and/or combinations thereof.

The one or more surfactants can also include one or more natural and/or naturally derived surfactants. Natural surfactants can include surfactants that are derived from natural products and/or surfactants that are minimally or not processed. Natural surfactants can include hydrogenated, non-hydrogenated, or partially hydrogenated vegetable oils, olus oil, Passiflora incarnata oil, candelilla cera, coco-caprylate, caprate, dicaprylyl ether, lauryl alcohol, myristyl myristate, dicaprylyl ether, caprylic acid, caprylic ester, octyl decanoate, octyl octanoate, undecane, tridecane, decyl oleate, oleic acid decylester, cetyl palmitate, stearic acid, palmitic acid, glyceryl stearate, hydrogenated, non-hydrogenated, or partially hydrogenated vegetable glycerides, Polyglyceryl-2 dipolyhydroxystearate, cetearyl alcohol, sucrose polystearate, glycerin, octadodecanol, hydrolyzed, partially hydrolyzed, or non-hydrolyzed vegetable protein, hydrolyzed, partially hydrolyzed, or non-hydrolyzed wheat protein hydrolysate, polyglyceryl-3 diisostearate, glyceryl oleate, myristyl alcohol, cetyl alcohol, sodium cetearyl sulfate, cetearyl alcohol, glyceryl laurate, capric triglyceride, coco-glycerides, lectithin, dicaprylyl ether, xanthan gum, sodium coco-sulfate, ammonium lauryl sulfate, sodium cocoyl sulfate, sodium cocoyl glutamate, polyalkylglucosides, such as decyl glucoside, cetearyl glucoside, cetyl stearyl polyglucoside, coco-glucoside, and lauryl glucoside, and/or combinations thereof. Natural surfactants can include any of the Natrue ingredients marketed by BASF, such as, for example, CegeSoft®, Cetiol®, Cutina®, Dehymuls®, Emulgade®, Emulgin®, Eutanol®, Gluadin®, Lameform®, LameSoft®, Lanette®, Monomuls®, Myritol®, Plantacare®, Plantaquat®, Platasil®, Rheocare®, Sulfopon®, Texapon®, and/or combinations thereof.

Other specific examples of surfactants include sodium lauryl sulfate, sodium lauryl isethionate, sodium lauroyl methyl isethionate, sodium cocoyl glutamate, sodium dodecyl benzene sulfonate, alkali metal or ammonium salts of lauroyl sarcosinate, myristoyl sarcosinate, palmitoyl sarcosinate, stearoyl sarcosinate and oleoyl sarcosinate, polyoxyethylene sorbitan monostearate, isostearate and laurate, sodium lauryl sulfoacetate, N-lauroyl sarcosine, the sodium, potassium, and ethanolamine salts of N-lauroyl, N-myristoyl, or N-palmitoyl sarcosine, polyethylene oxide condensates of alkyl phenols, cocoamidopropyl betaine, lauramidopropyl betaine, palmityl betaine, sodium cocoyl glutamate, and the like. Additional surfactants desired include fatty acid salts of glutamate, alkyl glucoside, salts of taurates, betaines, caprylates, and/or mixtures thereof. The oral care composition can also be sulfate free.

The oral care composition can comprise one or more surfactants each at a level from about 0.01% to about 15%, from about 0.3% to about 10%, or from about 0.3% to about 2.5%, by weight of the oral care composition.

### Thickening Agents

The oral care composition can comprise one or more thickening agents. Thickening agents can be useful in the oral care compositions to provide a gelatinous structure that stabilizes the dentifrice and/or toothpaste against phase separation. Suitable thickening agents include polysaccharides, polymers, and/or silica thickeners.

The thickening agent can comprise one or more polysaccharides. Some non-limiting examples of polysaccharides include starch; glycerite of starch; gums such as gum karaya (Sterculia gum), gum tragacanth, gum arabic, gum ghatti, gum acacia, xanthan gum, guar gum and cellulose gum; magnesium aluminum silicate (Veegum); carrageenan; sodium alginate; agar-agar; pectin; gelatin; cellulose compounds such as cellulose, microcrystalline cellulose, carboxymethyl cellulose, hydroxyethyl cellulose, hydroxypropyl cellulose, hydroxymethyl cellulose, hydroxymethyl carboxypropyl cellulose, methyl cellulose, ethyl cellulose, and sulfated cellulose; natural and synthetic clays such as hectorite clays; and mixtures thereof.

Other polysaccharides that are suitable for use herein include carageenans, gellan gum, locust bean gum, xanthan gum, carbomers, poloxamers, modified cellulose, and mixtures thereof. Carageenan is a polysaccharide derived from seaweed. There are several types of carageenan that may be distinguished by their seaweed source and/or by their degree of and position of sulfation. The thickening agent can comprise kappa carageenans, modified kappa carageenans, iota carageenans, modified iota carageenans, lambda carrageenan, and mixtures thereof. Carageenans suitable for use herein include those commercially available from the FMC Company under the series designation “Viscarin,” including but not limited to Viscarin TP 329, Viscarin TP 388, and Viscarin TP 389.

The thickening agent can comprise one or more polymers. The polymer can be a polyethylene glycol (PEG), a polyvinylpyrrolidone (PVP), polyacrylic acid, a polymer derived from at least one acrylic acid monomer, a copolymer of maleic anhydride and methyl vinyl ether, a crosslinked polyacrylic acid polymer, of various weight percentages of the oral care composition as well as various ranges of average molecular ranges. Alternatively, the oral care composition can be free of, essentially free of, or substantially free of a copolymer of maleic anhydride and methyl vinyl ether.

The thickening agent can comprise one or more inorganic thickening agents. Some non-limiting examples of suitable inorganic thickening agents include colloidal magnesium aluminum silicate, silica thickeners. Useful silica thickeners include, for example, include, as a non-limiting example, an amorphous precipitated silica such as ZEODENT® 165 silica. Other non-limiting silica thickeners include ZEODENT® 153, 163, and 167, and ZEOFREE® 177 and 265 silica products, all available from Evonik Corporation, and AEROSIL® fumed silicas.

The oral care composition can comprise from 0.01% to about 15%, from 0.1% to about 10%, from about 0.2% to about 5%, or from about 0.5% to about 2% of one or more thickening agents.

### Prenylated Flavonoids

The oral care composition of the present invention can comprise prenylated flavonoid. Flavonoids are a group of natural substances found in a wide range of fruits, vegetables, grains, bark, roots, stems, flowers, tea, and wine. Flavonoids can have a variety of beneficial effects on health, such as antioxidative, anti-inflammatory, antimutagenic, anticarcinogenic, and antibacterial benefits. Prenylated flavonoids are flavonoids that include at least one prenyl functional group (3-methylbut-2-en-1-yl, as shown in Formula VIII), which has been previously identified to facilitate attachment to cell membranes. Thus, while not wishing to being bound by theory, it is believed that the addition of a prenyl group, i.e. prenylation, to a flavonoid can increase the activity of the original flavonoid by increasing the lipophilicity of the parent molecule and improving the penetration of the prenylated molecule into the bacterial cell membrane. Increasing the lipophilicity to increase penetration into the cell membrane can be a double-edged sword because the prenylated flavonoid will tend towards insolubility at high Log P values (high lipophilicity). Log P can be an important indicator of antibacterial efficacy.

As such, the term prenylated flavonoids can include flavonoids found naturally with one or more prenyl functional groups, flavonoids with a synthetically added prenyl functional group, and/or prenylated flavonoids with additional prenyl functional groups synthetically added.

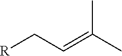

Formula VIII. Prenyl Function Group with R representing the other portions of the molecule

Other suitable functionalities of the parent molecule that improve the structure-activity relationship (e.g., structure-MIC relationship) of the prenylated molecule include additional heterocycles containing nitrogen or oxygen, alkylamino chains, or alkyl chains substituted onto one or more of the aromatic rings of the parent flavonoid.

Flavonoids can have a 15-carbon skeleton with at least two phenyl rings and at least one heterocyclic ring. Some suitable flavonoid backbones can be shown in Formula IX (flavone backbone), Formula X (isoflavan backbone), and/or Formula XI (neoflavonoid backbone).

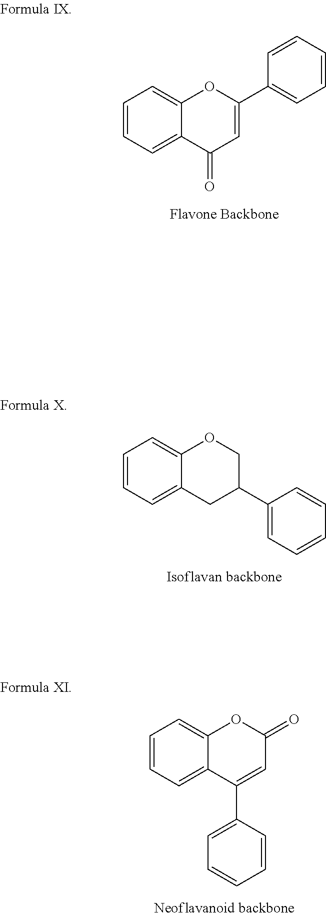 [SMILES: O=c1cc(-c2ccccc2)oc2ccccc12 ; c1ccc(C2COc3ccccc3C2)cc1 ; O=c1cc(-c2ccccc2)c2ccccc2o1]

Other suitable subgroups of flavonoids include anthocyanidins, anthoxanthins, flavanones, flavanonols, flavans, isoflavonoids, chalcones and/or combinations thereof.

Prenylated flavonoids can include naturally isolated prenylated flavonoids or naturally isolated flavonoids that are synthetically altered to add one or more prenyl functional groups through a variety of synthetic processes that would be known to a person of ordinary skill in the art of synthetic organic chemistry.

Other suitable prenylated flavonoids can include Bavachalcone, Bavachin, Bavachinin, Corylifol A, Epimedin A, Epimedin Al, Epimedin B, Epimedin C, Icariin, Icariside I, Icariside II, Icaritin, Isobavachalcone, Isoxanthohumol, Neobavaisoflavone, 6-Prenylnaringenin, 8-Prenylnaringenin, Sophoraflavanone G, (−)-Sophoranone, Xanthohumol, Quercetin, Macelignan, Kuraridin, Kurarinone, Kuwanon G, Kuwanon C, Panduratin A, 6-geranylnaringenin, Australone A, 6,8-Diprenyleriodictyol, dorsmanin C, dorsmanin F, 8-Prenylkaempferol, 7-O-Methylluteone, luteone, 6-prenylgenistein, isowighteone, lupiwighteone, and/or combinations thereof. Other suitable prenylated flavonoids include cannflavins, such as Cannflavin A, Cannflavin B, and/or Cannflavin C.

Preferably, the prenylated flavonoid has a high probability of having a MIC of less than about 25 ppm for S. aureus, a gram-positive bacterium. Suitable prenylated flavonoids include Bavachin, Bavachinin, Corylifol A, Icaritin, Isoxanthohumol, Neobavaisoflavone, 6-Prenylnaringenin, 8-Prenylnaringenin, Sophoraflavanone G, (−)-Sophoranone, Kurarinone, Kuwanon C, Panduratin A, and/or combinations thereof.

Preferably, the prenylated flavonoid has a high probability of having a MIC of less than about 25 ppm E. coli, a gram-negative bacterium. Suitable prenylated flavonoids include Bavachinin, Isoxanthohumol, 8-Prenylnaringenin, Sophoraflavanone G, Kurarinone, Panduratin A, and/or combinations thereof.

Approximately 1000 prenylated flavonoids have been identified from plants. According to the number of prenylated flavonoids reported before, prenylated flavonones are the most common subclass and prenylated flavanols is the rarest sub-class. Even though natural prenylated flavonoids have been detected to have diversely structural characteristics, they have a narrow distribution in plants, which are different to the parent flavonoids as they are present almost in all plants. Most of prenylated flavonoids are found in the following families, including Cannabaceae, Guttiferae, Leguminosae, Moraceae, Rutaceae and Umbelliferae. Leguminosae and Moraceae, due to their consumption as fruits and vegetables, are the most frequently investigated families and many novel prenylated flavonoids have been explored. Humulus lupulus of the Cannabaceae include 8-prenylnaringenin and xanthohumol, which can play a role in the health benefits of beer.

The prenylated flavonoid can be incorporated through a hops extract, incorporated in a separately added extract, or added as a separate component of the oral care compositions disclosed herein.

Suitable prenylated flavonoids can have a particular octanol-water partitioning coefficient. The octanol-water partitioning coefficient can be used to predict the lipophilicity of a compound. Without wishing to being bound by theory, it is believed that compounds that fall within the ranges described herein will be able to enter and/or disrupt the primarily hydrophobic phospholipid bilayer that makes of the cell membrane of microorganisms. Thus, the octanol-water partitioning coefficient can be correlated to the antibacterial effect of prenylated flavonoids. Suitable prenylated flavonoids can have a log P of at least about 2, at least about 4, from about 2 to about 10, from about 4 to about 10, from about 4 to about 7, or from about 4 to about 7.

The oral care composition can comprise at least about 0.001%, from about 0.001% to about 5%, from about 0.01% to about 2%, from about 0.0001% to about 2%, or at least about 0.05% of prenylated flavonoid.

### Other Ingredients

The oral care composition can comprise a variety of other ingredients, such as flavoring agents, sweeteners, colorants, preservatives, buffering agents, or other ingredients suitable for use in oral care compositions, as described below.

Flavoring agents also can be added to the oral care composition. Suitable flavoring agents include oil of wintergreen, oil of peppermint, oil of spearmint, clove bud oil, menthol, anethole, methyl salicylate, eucalyptol, cassia, 1-menthyl acetate, sage, eugenol, parsley oil, oxanone, alpha-irisone, marjoram, lemon, orange, propenyl guaethol, cinnamon, vanillin, ethyl vanillin, heliotropine, 4-cis-heptenal, diacetyl, methyl-para-tert-butyl phenyl acetate, and mixtures thereof. Coolants may also be part of the flavor system. Preferred coolants in the present compositions are the paramenthan carboxyamide agents such as N-ethyl-p-menthan-3-carboxamide (known commercially as “WS-3”) or N-(Ethoxycarbonylmethyl)-3-p-menthanecarboxamide (known commercially as “WS-5”), and mixtures thereof. A flavor system is generally used in the compositions at levels of from about 0.001% to about 5%, by weight of the oral care composition. These flavoring agents generally comprise mixtures of aldehydes, ketones, esters, phenols, acids, and aliphatic, aromatic and other alcohols. Sweeteners can be added to the oral care composition to impart a pleasing taste to the product.

Suitable sweeteners include saccharin (as sodium, potassium or calcium saccharin), cyclamate (as a sodium, potassium or calcium salt), acesulfame-K, thaumatin, neohesperidin dihydrochalcone, ammoniated glycyrrhizin, dextrose, levulose, sucrose, mannose, sucralose, stevia, and glucose.

Colorants can be added to improve the aesthetic appearance of the product. Suitable colorants include without limitation those colorants approved by appropriate regulatory bodies such as the FDA and those listed in the European Food and Pharmaceutical Directives and include pigments, such as TiO₂, and colors such as FD&C and D&C dyes.

Preservatives also can be added to the oral care compositions to prevent bacterial growth. Suitable preservatives approved for use in oral compositions such as methylparaben, propylparaben, benzoic acid, and sodium benzoate can be added in safe and effective amounts.

Titanium dioxide may also be added to the present composition. Titanium dioxide is a white powder which adds opacity to the compositions. Titanium dioxide generally comprises from about 0.25% to about 5%, by weight of the oral care composition.

Other ingredients can be used in the oral care composition, such as desensitizing agents, healing agents, other caries preventative agents, chelating/sequestering agents, vitamins, amino acids, proteins, other anti-plaque/anti-calculus agents, opacifiers, antibiotics, anti-enzymes, enzymes, pH control agents, oxidizing agents, antioxidants, and the like.

### Combinations

A. An array of anticavity oral care compositions, the array comprising:
(a) a first anticavity oral care composition comprising:

(i) a first subtherapeutic anticaries agent;

(ii) a second subtherapeutic anticaries agent; and

(b) a second anticavity oral care composition comprising a therapeutic anticaries agent.
B. The array as disclosed in A, wherein the first anticavity oral care composition is free of fluoride.
C. The array as disclosed in A or B, wherein the first subtherapeutic anticaries agent comprises hops, preferably wherein the hops comprises hops extract, Humulus lupulus extract, synthetically derived hops compounds, salts thereof, prodrugs thereof, or combinations thereof.
D. The array as disclosed in any of A to C, wherein the hops comprises hops alpha acid, hops iso-alpha acid, hops beta acid, or combinations thereof.
E. The array as disclosed in D, wherein the hops comprises hops beta acid, preferably wherein the hops beta acid comprises lupulone, adlupulone, colupulone, or combinations thereof.
F. The array as disclosed in D or E, wherein the hops comprises less than 1%, by weight of the hops, of hops alpha acid.
G. The array as disclosed in any of A to F, wherein the oral care composition comprises tin, zinc, calcium or combinations thereof, preferably wherein the oral composition comprises tin, zinc, or combinations thereof.
H. The array as disclosed in G, wherein the tin comprises stannous fluoride, stannous chloride, or combinations thereof.
I. The array as disclosed in G or H, wherein the zinc comprises zinc fluoride, zinc lactate, zinc oxide, zinc phosphate, zinc chloride, zinc acetate, zinc hexafluorozirconate, zinc sulfate, zinc tartrate, zinc gluconate, zinc citrate, zinc malate, zinc glycinate, zinc pyrophosphate, zinc metaphosphate, zinc oxalate, zinc carbonate, or combinations thereof.
J. The array as disclosed in any of G to I, wherein the calcium comprises calcium carbonate, calcium pyrophosphate, calcium phosphate, calcium citrate, calcium chloride, or combinations thereof.
K. The array as disclosed in any of D to J, wherein the first subtherapeutic anticaries agent comprises hops beta acid and the second subtherapeutic anticaries agent comprises stannous chloride.
L. The array as disclosed in any of A to J, wherein the second subtherapeutic anticaries agent comprises fluoride, preferably wherein the fluoride comprises stannous fluoride, sodium fluoride, sodium monofluorophosphate, amine fluoride, or combinations thereof.

### Examples

The invention is further illustrated by the following examples, which are not to be construed in any way as imposing limitations to the scope of this invention. Various other aspects, modifications, and equivalents thereof which, after reading the description herein, may suggest themselves to one of ordinary skill in the art without departing from the spirit of the present invention or the scope of the appended claims.

TABLE 3A
Compositions within Category A

| | SnF₂ | NaF | NaMFP |
|---|---|---|---|
| | Toothpaste | Toothpaste | Toothpaste |
| | Example 1-A | Example 2-A | Example 3-A |
| Ingredients | (wt %) | (wt %) | (wt %) |
| Sorbitol (70%) | 46.00 | 65.51 | — |
| USP Water | 22.70 | 11.60 | 56.30 |
| Sodium Fluoride | — | 0.24 | — |
| Stannous Fluoride | 0.45 | — | — |
| Sodium Monofluorophosphate | — | — | 0.76 |
| Stannous Chloride | 0.56 | — | — |
| Sodium Citrate | 1.55 | — | — |
| Sodium Gluconate | 1.30 | — | — |
| Trisodium phosphate | — | 1.10 | 0.42 |
| Monosodium phosphate | — | 0.42 | 0.08 |
| Tetrabasic Sodium | — | — | 0.60 |
| Pyrophosphate | | | |
| Hydrated Silica | 17.50 | 15.00 | 2.62 |
| Calcium Carbonate | — | — | 32.00 |
| Carbomer | — | 0.30 | — |
| Cellulose Gum | — | 0.75 | 0.92 |
| Carrageenan | 1.05 | — | 1.20 |
| Xanthan Gum, NF | 0.61 | — | — |
| Sodium Saccharin | 0.45 | 0.13 | 0.25 |
| Sodium lauryl sulfate | 5.00 | 3.60 | 4.00 |
| Sodium Hydroxide (50%) | 0.75 | — | — |
| Flavor | 1.20 | 0.80 | 0.85 |
| Titanium Dioxide | — | 0.55 | — |

TABLE 3B
Composition within Category A

| | Mouthrinse with NaF |
|---|---|
| | Example 4-A |
| Ingredients | (wt %) |
| USP Water | 99.13 |
| Propylene Glycol | 0.50 |
| Sodium Fluoride | 0.02 |
| Trisodium phosphate dodecahydrate | — |
| Monosodium phosphate monohydrate | — |
| Disodium Phosphate heptahydrate | 0.00 |
| Polysorbate 80 | 0.10 |
| Sodium Saccharin | — |
| Sodium Hydroxide (50%) | — |
| Flavor | 0.08 |
| Benzoic acid | 0.09 |
| Sodium benzoate | 0.02 |
| Phosphoric acid | 0.02 |
| Sucralose | 0.04 |

TABLE 3A and TABLE 3B provide exemplary compositions that fall within Category A. Suitable examples include compositions comprising a therapeutic dose of fluoride. TABLE 3A provides toothpaste compositions that combine fluoride, such as stannous fluoride, sodium fluoride, and/or sodium monofluorophosphate, with a variety of other ingredients, such as abrasives, thickening agents, metal ion sources, humectants, etc. TABLE 3B provides a mouth rinse example that can be within Category A (therapeutic dose of fluoride).

TABLE 4
Compositions within Category B

| | Example | Example | Example | Example |
|---|---|---|---|---|
| | 1-B | 2-B | 3-B | 4-B |
| | MFP/ | MFP/Hops | SnF2/ | NaF/ |
| | Hops | anhydrous | Hops | Hops |
| | (wt %) | (wt %) | (wt %) | (wt %) |
| Sorbitol (70%) | 36.83 | — | 46.00 | 65.51 |
| Glycerin | — | 47.7 | — | — |
| USP Water | 13 | — | 26.58 | 13.50 |
| Sodium Fluoride | — | — | — | 0.24 |
| Stannous Fluoride | — | — | 0.45 | — |
| Sodium | 1.15 | 1.15 | — | — |
| Monofluorophosphate | | | | |
| Stannous Chloride | 1.1 | 1.1 | 0.56 | — |
| Sodium Citrate | — | — | 1.55 | — |
| Sodium Gluconate | 1 | 1 | 1.30 | — |
| Trisodium phosphate | — | — | — | 1.10 |
| Monosodium phosphate | — | — | — | 0.42 |
| Hydrated Silica | — | — | 17.50 | 15.00 |
| Calcium Carbonate | 32 | 32 | — | — |
| Carbomer | — | 1 | — | 0.30 |
| Cellulose Gum | 1 | — | — | 0.75 |
| Carrageenan | — | — | 1.05 | — |
| Xanthan Gum, NF | 0.3 | 0.5 | 0.61 | — |
| Sodium Saccharin | 0.5 | 0.40 | 0.45 | 0.13 |
| Sodium lauryl sulfate | 1.29 | 1.40 | 1.50 | 1.20 |
| Stevia Glycosides | — | 0.30 | — | — |
| Sodium Hydroxide | 0.33 | 1.35 | 0.75 | — |
| (50%) | | | | |
| Hops Beta Extract | 0.50 | 0.50 | 0.50 | 0.50 |
| Flavor | 1 | 1.10 | 1.20 | 0.80 |
| Titanium Dioxide | — | 0.5 | — | 0.55 |
| Sodium bicarbonate | 10 | 10 | — | — |

TABLE 4 displays compositions that fall within Category B, which are compositions that have a therapeutic dose of fluoride and a subtherapeutic dose of a second anticaries agent, such as hops.

TABLE 5
Compositions with Category C

| | Example | Example | Example |
|---|---|---|---|
| | 1-C | 2-C | 3-C |
| | MFP/Hops | SnF2/Hops | NaF/Hops |
| Ingredients | (wt %) | (wt %) | (wt %) |
| Sorbitol (70%) | 36.98 | 46.12 | 65.75 |
| USP Water | 13 | 26.80 | 13.50 |
| Sodium Fluoride | — | — | 0.06 |
| Stannous Fluoride | — | 0.11 | — |
| Sodium | 0.19 | — | — |
| Monofluorophosphate | | | |
| Stannous Chloride | 1.1 | 0.56 | — |
| Sodium Citrate | — | 1.55 | — |
| Sodium Gluconate | 1 | 1.30 | |
| Trisodium phosphate | — | — | 1.10 |
| Monosodium phosphate | — | — | 0.42 |
| Tetrabasic | — | — | — |
| Sodium Pyrophosphate | | | |
| Hydrated Silica | — | 17.50 | 15.00 |
| Calcium Carbonate | 32 | — | — |
| Carbomer | — | — | 0.30 |
| Cellulose Gum | 1 | — | 0.75 |
| Carrageenan | — | 1.05 | — |
| Xanthan Gum, NF | 0.3 | 0.61 | — |
| Sodium Saccharin | 0.5 | 0.45 | 0.13 |
| Sodium lauryl sulfate | 1.29 | 1.50 | 1.20 |
| Stevia Glycosides | — | — | — |
| Sodium Hydroxide (50%) | 0.33 | 0.75 | — |
| Hops Beta Acid Extract | 0.5 | 0.50 | 0.50 |
| Flavor | 1 | 1.20 | 0.80 |
| Titanium Dioxide | — | — | 0.55 |
| Sodium bicarbonate | 10 | — | — |

TABLE 5 displays compositions that fall within Category C, which are compositions that have a subtherapeutic dose of fluoride and a subtherapeutic dose of a second anticaries agent, such as hops, but collectively has a therapeutic anticaries and/or anticavity benefit.

TABLE 6
Compositions with Category D

| | | Example | Example | Example |
|---|---|---|---|---|
| | Example | 2-D | 3-D | 4-D |
| | 1-D | SnCl₂/ | SnCl₂/ | SnCl₂/ |
| | SnCl₂/ | Hops | Hops/ | Hops/ |
| | Hops | anhydrous | Silica | Bicarb |
| | (wt %) | (wt %) | (wt %) | (wt %) |
| Sorbitol (70%) | 36.83 | — | 44.00 | 36.00 |
| Glycerin | — | 47.7 | — | — |
| USP Water | 13 | — | 28.99 | 26.99 |
| Sodium Fluoride | — | — | — | — |
| Stannous Fluoride | — | — | — | — |
| Sodium | — | — | — | — |
| Monofluorophosphate | | | | |
| Stannous Chloride | 1.1 | 1.1 | 1.10 | 1.10 |
| Sodium Citrate | — | — | 1.05 | 1.05 |
| Sodium Gluconate | 1 | 1 | 1.30 | 1.30 |
| Hydrated Silica | — | — | 17.50 | 17.50 |
| Calcium Carbonate | 32 | 32 | — | — |
| Carbomer | — | 1 | — | — |
| Cellulose Gum | 1 | | — | — |
| Carrageenan | — | — | 1.05 | 1.05 |
| Xanthan Gum, NF | 0.3 | 0.5 | 0.61 | 0.61 |
| Sodium Saccharin | 0.5 | 0.40 | 0.45 | 0.45 |
| Sodium lauryl sulfate | 1.29 | 1.40 | 1.50 | 1.50 |
| Stevia Glycosides | — | 0.30 | — | — |
| Sodium Hydroxide | 0.33 | 1.35 | 0.75 | 0.75 |
| (50%) | | | | |
| Hops Beta Acid Extract | 0.5 | 0.5 | 0.50 | 0.50 |
| Flavor | 1 | 1.10 | 1.20 | 1.20 |
| Titanium Dioxide | — | 0.5 | — | — |
| Sodium bicarbonate | 10 | 10 | — | 10.00 |

TABLE 6 displays compositions that fall within Category D, which are compositions that have a subtherapeutic dose of a first anticaries agent and a subtherapeutic dose of a second anticaries agent, but collectively has a therapeutic anticaries and/or anticavity benefit.

TABLE 7
Hops Beta Acids Extract Specification

| Ingredient | Amount (wt %) |
|---|---|
| Hops Beta Acids | 45 ± 2 |
| Hops Alpha Acids | 0.4 ± 0.3 |
| Hops oils | 1.5 ± 0.5 |
| Propylene Glycol | 20 ± 15 |
| Water | <8% |
| pH | 11 ± 0.5 |

TABLE 7 describes the hops beta acid extract provided by Hopsteiner®. Since the hops beta acids are provided as an extract, there can be some variability in the amounts of certain ingredients. However, the extract comprises approximately 45%, by weight of the extract, of the hops beta acids and approximately 0.4%, by weight of the extract, of hops alpha acids. This is dramatically different to previous hops extracts which typically have more hops alpha acids than hops beta acids. Other minor ingredients may be present in the Hops Beta Acid extract.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An array of anticavity oral care compositions, the array comprising:
(a) a first anticavity oral care composition comprising:
(i) a first subtherapeutic anticaries agent;
(ii) a second subtherapeutic anticaries agent; and
(b) a second anticavity oral care composition comprising a therapeutic anticaries agent.

2. The array of claim 1, wherein the first anticavity oral care composition is free of fluoride.

3. The array of claim 1, wherein the first subtherapeutic anticaries agent comprises hops.

4. The array of claim 3, wherein the hops comprises hops alpha acid, hops beta acid, or combinations thereof.

5. The array of claim 4, wherein the hops comprises hops beta acid.

6. The array of claim 5, wherein the hops comprises less than 1%, by weight of the hops, of hops alpha acid.

7. The array of claim 3, wherein the hops comprises hops extract.

8. The array of claim 1, wherein the second subtherapeutic agent comprises calcium, tin, zinc, or combinations thereof.

9. The array of claim 8, wherein the tin comprises stannous fluoride, stannous chloride, or combinations thereof.

10. The array of claim 8, wherein the calcium comprises calcium abrasive, calcium salt, or combinations thereof.

11. The array of claim 10, wherein the calcium comprises calcium carbonate, calcium pyrophosphate, calcium phosphate, calcium citrate, calcium chloride, or combinations thereof.

12. The array of claim 10, wherein the zinc comprises zinc fluoride, zinc lactate, zinc oxide, zinc phosphate, zinc chloride, zinc acetate, zinc hexafluorozirconate, zinc sulfate, zinc tartrate, zinc gluconate, zinc citrate, zinc malate, zinc glycinate, zinc pyrophosphate, zinc metaphosphate, zinc oxalate, zinc carbonate, or combinations thereof.

13. The array of claim 2, wherein the first subtherapeutic anticaries agent comprises hops beta acid and the second subtherapeutic anticaries agent comprises stannous chloride.

14. The array of claim 1, wherein the second subtherapeutic anticaries agent comprises fluoride.

15. The array of claim 14, wherein the fluoride comprises stannous fluoride, sodium fluoride, sodium monofluorophosphate, amine fluoride, or combinations thereof.

16. An array of anticavity oral care compositions, the array comprising:
(a) a first anticavity oral care composition comprising:
(i) a first therapeutic anticaries agent comprising fluoride; and
(ii) a first subtherapeutic anticaries agent, wherein the first oral care composition has a therapeutic anticavity benefit greater than the first therapeutic anticaries agent; and
(b) a second oral care composition comprising a second therapeutic anticaries agent, wherein the therapeutic anticavity benefit of the first oral care composition is greater than the therapeutic anticavity benefit of the second oral care composition.

17. The array of claim 16, wherein the fluoride comprises stannous fluoride, sodium fluoride, sodium monofluorophosphate, amine fluoride, or combinations thereof.

18. The array of claim 16, wherein the first therapeutic anticaries agent comprises calcium, tin, zinc, or combinations thereof.

19. The array of claim 18, wherein the tin comprises stannous fluoride, stannous chloride, or combinations thereof.

20. The array of claim 18, wherein the calcium comprises calcium abrasive, calcium salt, or combinations thereof.

21. The array of claim 20, wherein the calcium comprises calcium carbonate, calcium pyrophosphate, calcium phosphate, calcium citrate, calcium chloride, or combinations thereof.

22. The array of claim 18, wherein the zinc comprises zinc fluoride, zinc lactate, zinc oxide, zinc phosphate, zinc chloride, zinc acetate, zinc hexafluorozirconate, zinc sulfate, zinc tartrate, zinc gluconate, zinc citrate, zinc malate, zinc glycinate, zinc pyrophosphate, zinc metaphosphate, zinc oxalate, zinc carbonate, or combinations thereof.

23. The array of claim 16, wherein the first subtherapeutic anticaries agent comprises hops.

24. The array of claim 23, wherein the hops comprises hops alpha acid, hops beta acid, or combinations thereof.

25. The array of claim 24, wherein the hops comprises hops extract.

26. The array of claim 25, wherein the hops extract has less than about 1%, by weight of the hops extract, of hops alpha acid.

27. The array of claim 16, wherein the second therapeutic anticaries agent comprises fluoride.

28. The array of claim 27, wherein the fluoride comprises stannous fluoride, sodium fluoride, sodium monofluorophosphate, amine fluoride, or combinations thereof.

29. The array of claim 16, wherein the array comprises a third anticavity oral care composition, the third anticavity oral care composition comprises:
(i) a second subtherapeutic anticaries agent; and
(ii) a third subtherapeutic anticaries agent,
wherein the third oral care composition has a therapeutic anticavity benefit less than the therapeutic anticavity benefit of the first oral care composition.

30. The array of claim 29, wherein the third anticavity oral care composition is free of fluoride.

31. The array of claim 29, wherein the second subtherapeutic anticaries agent comprises hops.

32. The array of claim 31, wherein the hops comprises hops alpha acid, hops beta acid, or combinations thereof.

33. The array of claim 31, wherein the hops comprises hops beta acid.

34. The array of claim 32, wherein the hops comprises less than 1%, by weight of the hops, of hops alpha acid.

35. The array of claim 31, wherein the hops comprises hops extract.

36. The array of claim 29, wherein the third subtherapeutic agent comprises calcium, tin, zinc, or combinations thereof.

37. The array of claim 36, wherein the tin comprises stannous fluoride, stannous chloride, or combinations thereof.

38. The array of claim 36, wherein the calcium comprises calcium abrasive, calcium salt, or combinations thereof.

39. The array of claim 38, wherein the calcium comprises calcium carbonate, calcium pyrophosphate, calcium phosphate, calcium citrate, calcium chloride, or combinations thereof.

40. The array of claim 36, wherein the zinc comprises zinc fluoride, zinc lactate, zinc oxide, zinc phosphate, zinc chloride, zinc acetate, zinc hexafluorozirconate, zinc sulfate, zinc tartrate, zinc gluconate, zinc citrate, zinc malate, zinc glycinate, zinc pyrophosphate, zinc metaphosphate, zinc oxalate, zinc carbonate, or combinations thereof.

41. The array of claim 29, wherein the second subtherapeutic anticaries agent comprises hops beta acid and the third subtherapeutic anticaries agent comprises stannous chloride.
